(19) United States
(12) Patent Application Publication
(10) Pub. No.: US 2026/0204338 A1
(43) Pub. Date: Jul. 16, 2026

(54) FRAGMENT-BASED QUANTUM MECHANICAL CALCULATION OF PROTEIN PROPERTIES

(71) Applicant: Microsoft Technology Licensing, LLC, Redmond, WA (US)

(72) Inventors: Tong WANG, Beijing (CN); Bin SHAO, Beijing (CN); Tieyan LIU, Beijing (CN)

(73) Assignee: Microsoft Technology Licensing, LLC, Redmond, WA (US)

(21) Appl. No.: 19/137,085

(22) PCT Filed: Dec. 21, 2022
(86) PCT No.: PCT/CN2022/140662

§ 371 Date: Jun. 9, 2025

(51) Int. Cl. G16B 5/00; G06N 10/80; G16B 40/00

(52) U.S. Cl. CPC G16B 5/00; G06N 10/80; G16B 40/00

(57) ABSTRACT

A computing system for fragment-based quantum mechanical calculation of protein properties is provided. A processor implements a protein fragmentation module that separates a computer-readable polypeptide sequence into a plurality of data units. For each subsequence of three adjacent amino acids in the polypeptide sequence, a first amino acid, a second amino acid, and a third amino acid are identified, each amino acid having a respective main chain including an amino group, a carbon, and a carboxyl group, and a side chain attached to the alpha carbon. The protein fragmentation module generates a data unit representing a first alpha carbon, a first carboxyl group, a second amino group, a second alpha carbon, a second carboxyl group, a second side chain, a third amino group, and a third alpha carbon, and stores the generated data unit in the memory.

### BACKGROUND

In the field of computational chemistry, computer-based techniques have been developed to predict molecular properties through computer simulations. These molecular properties can have a wide-ranging impact on the appearance and function of a molecule or material, and thus are of keen interest in a wide variety of fields. For example, in the field of drug design, changes in molecular properties can affect the efficacy of a drug. In the field of drug discovery, molecular properties can affect the potential for a material found in nature to be used for therapeutic purposes. In the field of quantum chemistry, quantum-mechanical calculation of electronic contributions to physical and chemical properties of molecules and materials is a fundamental area of inquiry. As discussed below, opportunities remain for improvements in computational methods for predicting molecular properties, which would have application beyond the field of computational chemistry.

### SUMMARY

To address the issues discussed herein, computerized systems and methods for fragment-based quantum mechanical calculation of protein properties are provided. In one aspect, the computerized system includes a processor that executes instructions using portions of associated memory to implement a protein fragmentation module. The protein fragmentation module separates a computer-readable polypeptide sequence representing a plurality of amino acids into a plurality of data units. For each subsequence of three adjacent amino acids in the polypeptide sequence, the protein fragmentation module is configured to identify a first amino acid, identify a second amino acid, identify a third amino acid, generate a data unit, and store the generated data unit. The first amino acid has a first main chain comprising a first amino group, a first alpha carbon, and a first carboxyl group, and a first side chain attached to the first alpha carbon. The second amino acid has a second main chain comprising a second amino group, a second alpha carbon, and a second carboxyl group, and a second side chain attached to the second alpha carbon. The third amino acid has a third main chain comprising a third amino group, a third alpha carbon, and a third carboxyl group, and a third side chain attached to the third alpha carbon. The data unit comprises data representing the first alpha carbon, the first carboxyl group, the second amino group, the second alpha carbon, the second carboxyl group, the second side chain, the third amino group, and the third alpha carbon.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows a schematic view of a computing system for fragment-based quantum mechanical calculation of protein properties, according to one embodiment of the present disclosure.

FIG. 2 shows generation of data units from a truncated polypeptide sequence, using the computing system of FIG. 1.

FIG. 3 shows generation of data units from a tetrapeptide, using the computing system of FIG. 1.

FIG. 4 shows a subset of atoms that coexist in the data units generated from the tetrapeptide of FIG. 3.

FIG. 5 shows atoms that coexist in the data units generated from the tetrapeptide of FIG. 3, as well as an indication of redundant units.

FIG. 6 shows coexisting atoms pairs for each atom in the tetrapeptide of FIG. 3 that will be included in the force calculation of the polypeptide.

FIG. 7 shows atoms in the tetrapeptide of FIG. 3 for which extra interactions need to be calculated by molecular mechanics.

FIG. 8 shows atoms in the tetrapeptide of FIG. 3 for which extra interactions need to be calculated by a combination of quantum mechanics and molecular mechanics.

FIG. 9 shows a flowchart of a method for fragment-based quantum mechanical calculation of protein properties, according to an example implementation of the present disclosure.

FIG. 10 shows an example computing environment according to which the embodiments of the present disclosure may be implemented.

### DETAILED DESCRIPTION

Computer-based techniques have been developed to predict molecular properties through computer simulations. For example, molecular dynamics (MD) simulation is a widely used computational tool that simulates the movements of atoms. MD models compute potential energies and resultant atomic forces at each atom of a molecular system as the atoms change physical position over a simulation time period, to thereby describe kinetic and thermodynamic properties of the molecular system. MD is widely used in the physical, chemical, biological, and pharmaceutical fields, as understanding the mechanisms of protein molecules enables advancements in drug design, protein design, enzyme engineering, and the like.

MD simulations can be performed using classic molecular mechanics (MM) or quantum mechanics (QM). Classic MM is based on Newtonian mechanics and has been widely used for proteins. Classic MM simulations employing empirical force fields can achieve fast simulation results for large systems, but suffer from the drawback of failing to capture the quantum effect caused by electron movement. Additionally, the parameters of the force fields computed within such simulations are not typically transferable.

In contrast, QM provides highly accurate calculations for atoms and molecules, and can thus be used to study biological processes with electron transitions. Density Function Theory (DFT) is the most widely used approach in quantum simulation. DFT is a powerful quantum physics calculation technique that can in many cases accurately predict various molecular properties such as energy and forces of molecules, the shape of molecules, etc. While MD simulations driven by DFT can accurately calculate energy and forces, DFT is time-consuming and computationally intensive, often taking up to several hours for a single model of a simple molecule on a conventional processor, and months for simulation of a protein comprised of 1000 or more atoms. As such, for complex molecular systems, computing precise DFT solutions is not practical on current hardware. These factors present a barrier to accurately and efficiently predicting molecular properties of proteins.

To address these issues, a computing system for fragment-based quantum mechanical calculation of protein properties is provided. While it is computationally prohibitive to run QM directly for biomolecules, applying a hybrid strategy using QM and classic MM enable a more efficient and more accurate determination of forces on each atom in a polypeptide sequence, i.e., protein. The embodiments discussed herein describe a novel approach using polypeptide fragments, i.e., data units, to calculate the molecular properties of a protein using a combination of QM and classic MM.

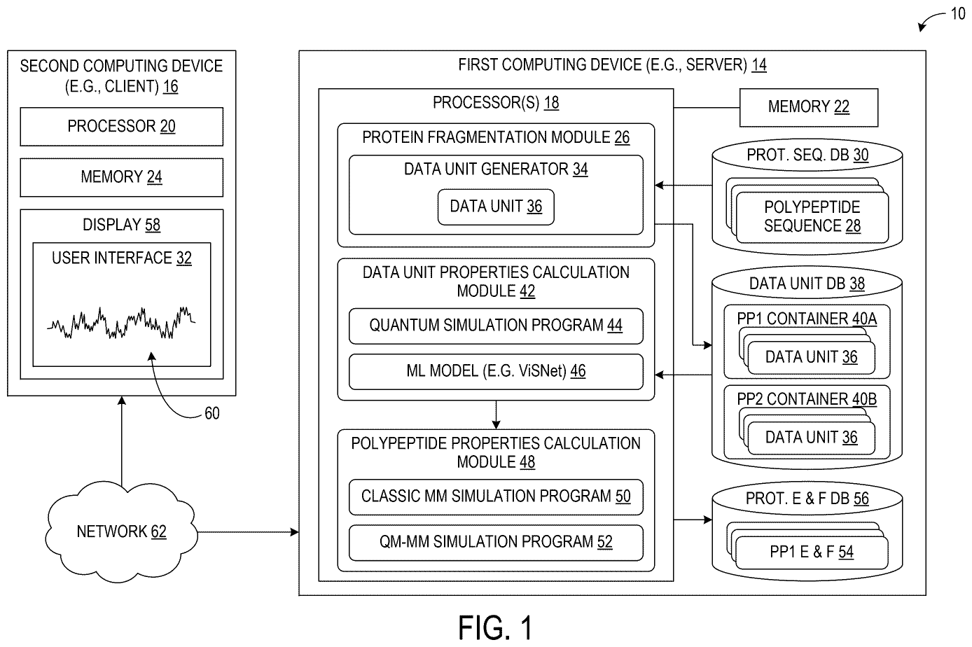

Referring initially to FIG. 1, the computing system 10 includes at least one computing device. The computing system 10 is illustrated as including a first computing device 14 including a processor 18 and memory 22, and a second computing device 16 including a processor 20 and memory 24. The illustrated implementation is exemplary in nature, and other configurations are possible. In the description below, the first computing device will be described as a server 14 and the second computing device will be described as a client computing device 16, and respective functions carried out at each device will be described. It will be appreciated that in other configurations, the computing system 12 may include a single computing device that carries out the salient functions of both the server 14 and client computing device 16, and that the first computing device could be a computing device other than server. In other alternative configurations, functions described as being carried out at the server 14 may alternatively be carried out at the client computing device 16 and vice versa.

Continuing with FIG. 1, the processor 18 is configured to implement a protein fragmentation module 26 hosted at the server 14. The protein fragmentation module 26 separates a computer-readable polypeptide sequence 28 representing a plurality of amino acids into a plurality of data units. The polypeptide sequence 28 may be stored at a protein sequence database 30, such as UniProt, Swiss-Prot, protein research foundation (PRF), and the like, and sent to the protein fragmentation module 26 upon receiving user input via a user interface 32 at the client computing device 16. It will be appreciated that a polypeptide chain of one hundred or more amino acids linked together via covalent peptide bonds is generally considered a protein. In the embodiments described herein, the computing system 10 is configured to determine the forces and energy for proteins, as well as for polypeptides comprised of fewer than one hundred amino acids.

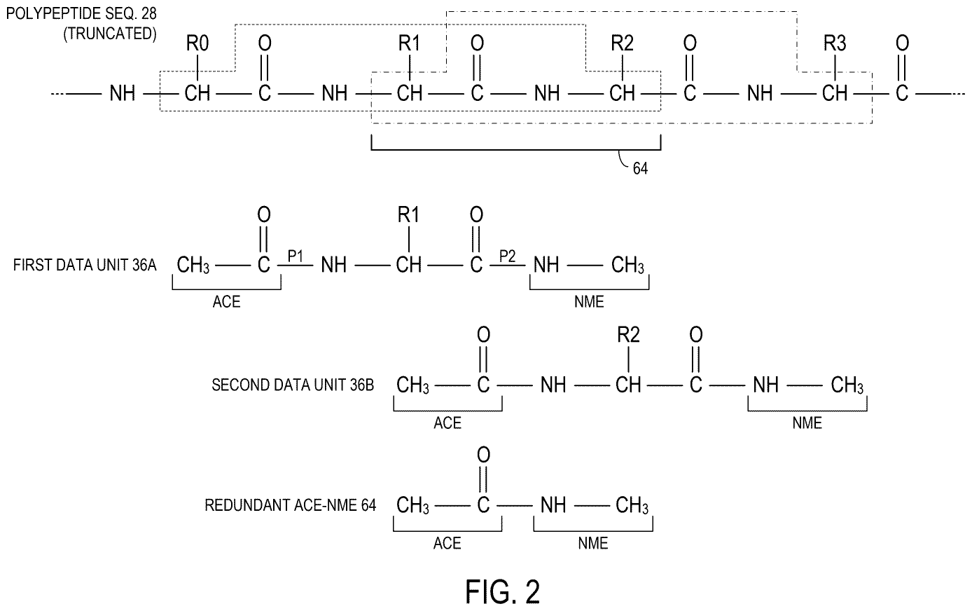

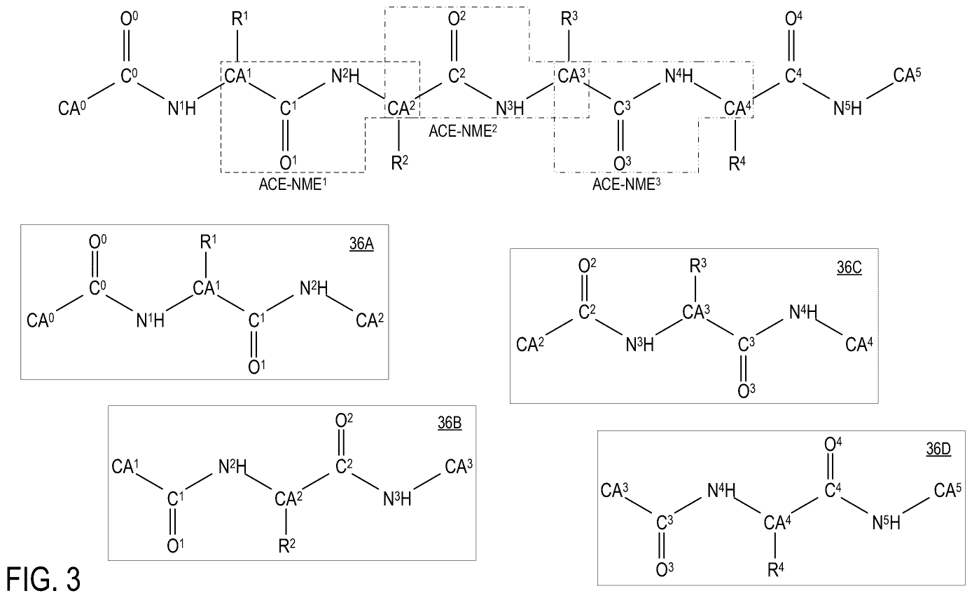

The amino acids in the polypeptide sequence 28 may be represented in a single letter code (e.g., ALGY for alanine, leucine, glycine, and tyrosine) or a three letter code (e.g., AlaLeuGlyTyr for alanine, leucine, glycine, and tyrosine). As discussed in detail below with reference to FIGS. 2 and 3, a data unit generator 34 included in the protein fragmentation module 26 is configured to separate the polypeptide sequence 28 into a plurality of data units 36, each data unit 36 representing the atomic structure of a subsequence of amino acids in the polypeptide sequence 28. The plurality of data units 36 may be stored on the server 14 in a data unit database 38. It will be appreciated that the data unit database 38 may include multiple containers 40 that each store data units 36 derived from a respective polypeptide sequence 28.

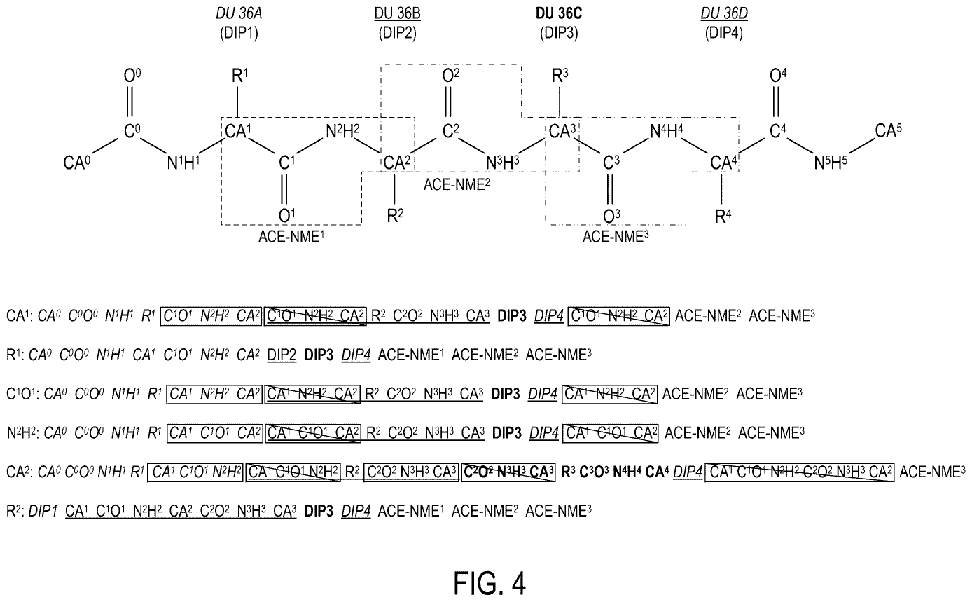

The processor 18 is further configured to implement a data unit properties calculation module 42 that calculates a force F of each atom in each data unit 36 of the plurality of data units, and calculates an energy E of each data unit 36. As shown in FIG. 1 and discussed in detail below with reference to FIGS. 4-6, the data unit properties calculation 40 may include a quantum simulation program 44 that applies DFT to calculate the force of each atom in each data unit 36, and to calculate the energy of each data unit 36. Alternatively, in some implementations, the force of each atom in each data unit 36 and the energy of each data unit 36 may be determined via a machine learning (ML) model 46, such as a Vector-Scalar interactive Graph Neural Network (ViSNet), for example.

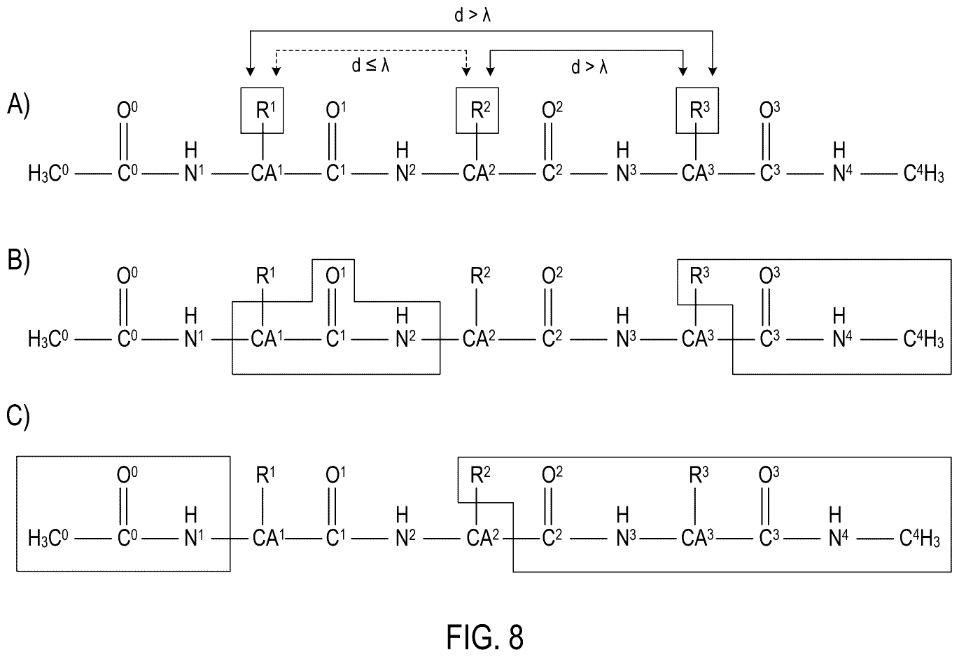

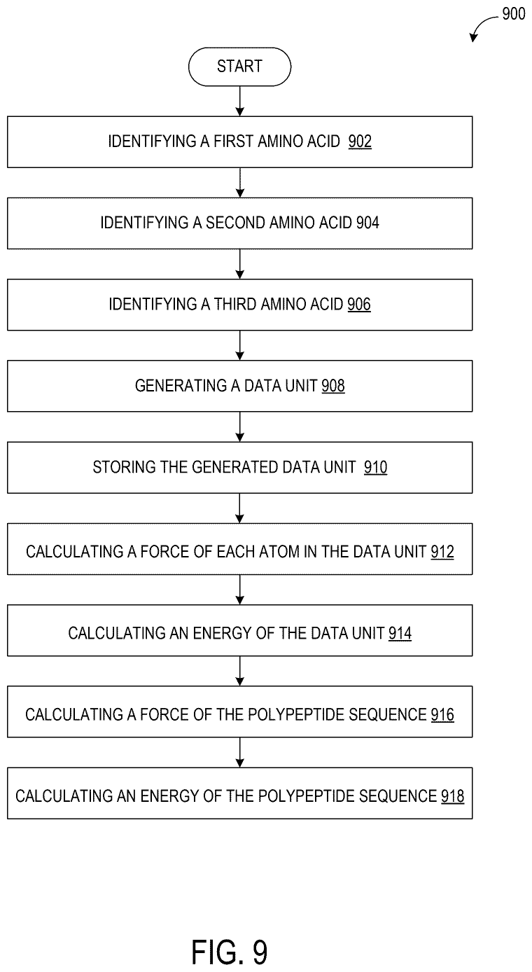

Continuing with FIG. 1, the processor 18 is further configured to implement a polypeptide properties calculation module 48 that calculates a force of the polypeptide sequence 28 based on the calculated forces of each atom in each data unit 26 of the plurality of data units, and calculates an energy of the polypeptide sequence based on the calculated energies for each data unit of the plurality of data units. As described in detail below with reference to FIGS. 8 and 9, the energy of the polypeptide sequence 28 is calculated by summing the calculated energy of each data unit 26 of the plurality of data units and subtracting energies of duplicated regions shared by adjacent data units of the polypeptide sequence. Similarly, the force of the polypeptide sequence 28 is calculated by summing the calculated force of each data unit 26 of the plurality of data units and subtracting forces of duplicated regions shared by adjacent data units of the polypeptide sequence. The polypeptide properties calculation module 48 includes a classic MM simulation program 50 to calculate interactions between main chain atoms of a data unit 26 and side chain atoms of non-adjacent data units. The polypeptide properties calculation module 48 further includes a hybrid QM-MM simulation program 52. This program enables interactions between side chain atoms of data units separated by a distance that is less than or equal to a distance threshold to be calculated via counterpoise QM applying DFT, while interactions between side chain atoms of data units separated by a distance that is greater than the distance threshold are calculated via classic MM.

Once determined, the calculated energy and force 54 for each polypeptide sequence 28 may be stored on the server 14 in a protein energy and force database 56. In response to a user input at the client computing device 16, the energy and force for the polypeptide sequence may be displayed on a display 58 in the user interface 32 as a graph 60. In any of the implementations described herein, it will be appreciated the server 14 is in communication with the client computing device 16 via a network 62, which allows a user of the client computing device to access data and programs stored on the server 14, including data stored in the protein sequence database 30, the data unit database 38, and the protein energy and force database 56.

### Protein Fragmentation

Each amino acid includes a main chain with an amino group (NH₂), an alpha carbon (Cα), and a carboxyl group (COOH), as well as a side chain R attached to the alpha carbon. It is generally accepted that there are twenty-one amino acid side chains, each of which determines the identity of the amino acid. When forming a polypeptide chain, the amino group of a downstream amino acid forms a peptide bond with the carboxyl group of an upstream amino acid in a biochemical reaction that releases a molecule of water. Amino acid sequences are read from left to right, with the first amino group forming a N-terminus at the beginning of the sequence and the last carboxyl group forming a C-terminus at the end of the sequence.

For each subsequence of three adjacent amino acids in the polypeptide sequence 28, the data unit generator 34 is configured to identify a first amino acid, a second amino acid, and a third amino acid. The first amino acid has a first main chain comprising a first amino group, a first alpha carbon, and a first carboxyl group, and a first side chain attached to the first alpha carbon. The second amino acid has a second main chain comprising a second amino group, a second alpha carbon, and a second carboxyl group, and a second side chain attached to the second alpha carbon. The third amino acid has a third main chain comprising a third amino group, a third alpha carbon, and a third carboxyl group, and a third side chain attached to the third alpha carbon. The data unit generator 34 is configured to generate a data unit 36 comprising data representing the first alpha carbon, the first carboxyl group, the second amino group, the second alpha carbon, the second carboxyl group, the second side chain, the third amino group, and the third alpha carbon.

An example of two generated data units 26A, 26B are shown in FIG. 2. As illustrated, a truncated polypeptide sequence 28 is separated into a first data unit 36A, indicated by the dashed line, and a second data unit 36B, indicated by the dash-dot line. The first alpha carbon and the first carboxyl group of each data unit 36 comprise an N-terminal acetyl group (ACE) of the data unit, and the third amino group and third alpha carbon comprise a C-terminal N-methylamino group (NME) of the data unit. Each data unit 36 further includes a first peptide bond P1 formed between the N-terminal ACE and the second amino group, and a second peptide bond P2 formed between the second carboxyl group and the C-terminal NME. With two peptide bonds, each data unit 36 can be considered a novel type of dipeptide (DIP).

A region of overlap between the first and second data units 36A, 36B in the truncated polypeptide sequence 28 is indicated by a bracket. The region of overlap includes the N-terminal ACE from the second data unit 36B and the C-terminal NME of first the data unit 36A. As discussed in detail below, when calculating the forces and energy for the polypeptide sequence, force and energy for each region of overlap, i.e., redundant ACE-NME unit 64, must be subtracted from the equation.

For each data unit 36, data representing one or more additional hydrogens is added to the first alpha carbon in each data unit 36 according to a first bond length and a first direction of a previous bond between the first alpha carbon and the first side chain. Additionally, data representing one or more additional hydrogens is added to the third alpha carbon in each data unit according to a third bond length and a third direction of a previous bond between the third alpha carbon and the third side chain. A limited-memory Broyden-Fletcher-Goldfarb-Shanno quasi-Newton (LBFGS) algorithm is applied to optimize the position of the one or more additional hydrogens.

FIG. 3 illustrates a tetrapeptide separated into four data units 36A, 36B, 36C, 36D. The four data units are shown individually in boxes. Each data unit includes a main chain with an amino group (N^{x}H), an alpha carbon (CA^{x}), and a hydroxyl group (C^{x}O^{x}), with a side chain R^{x} attached to the alpha carbon. The alpha carbon and hydroxyl group from the upstream amino acid in the polypeptide sequence comprise an ACE cap at the N-terminus, and the amino group and alpha carbon from the downstream amino acid comprise an NME cap at the C-terminus. For example, in data unit 26A, the main chain includes N¹H, CA¹, and C¹O¹. The side chain R¹ is attached to the alpha carbon CA¹. The upstream alpha carbon CA⁰ and hydroxyl group C⁰O⁰ form the N-terminal ACE cap, and the downstream amino group N²H and alpha carbon CA² form the C-terminal NME cap. Separation of the tetrapeptide into four data units yields three redundant ACE-NME units, indicated in FIG. 3 by dashed line, dash-dot line, and dash-dot-dot line.

A data unit 36 is generated for each amino acid represented in the polypeptide sequence 28 and stored in the data unit database 38. The energy and force that are necessary for determining a force field of the polypeptide sequence are then calculated for each data unit 36. The generalized force field consists of two parts: energy and force calculation for each data unit 36, and two-body interaction calculation between nearby data units 36. The total energy and force for the whole polypeptide, i.e., protein, can be precisely determined from these two aspects.

### Data Unit Properties Calculation

The following paragraphs provide additional description of implementations for calculating the molecular properties of individual data units 36. As discussed above, there are two different ways to calculate the molecular properties of data units, including quantum mechanics (QM) based on ORCA and a deep learning (DL) model.

As described above, the data unit properties calculation module 42 includes a quantum simulation program 44. In a quantum mechanical (QM) mode, the quantum simulation program 44 applies density functional theory (DFT) to calculate the force of each atom in each generated data unit 36, and to calculate the energy of each data unit. An example implementation of such a QM program is ORCA, a general-purpose quantum chemistry program package that includes modern electronic structure methods, such as DFT. Using ORCA, a DFT such as the M06-2X density functional, is applied to a basis set, such as the 6-31G (d) basis set, to calculate the force for each atom and energy for the data unit 36. The M06-2X functional is a high-nonlocality functional with double the amount of nonlocal exchange (2X), and it is parametrized only for nonmetals. In the 6-31G basis set, each inner shell (1 s orbital) STO is a linear combination of 6 primitives and each valence shell STO is split into an inner and outer part (double zeta) using 3 and 1 primitive Gaussians, respectively.

As described above, a redundant ACE-NME unit 64 between adjacent amino acids must be accounted for when combining the data units 36 to determine the total energy of the polypeptide sequence. Thus, the total energy of the whole protein can be approximately calculated by the sum of the energies of the data units 36 and subtracting the energies of all redundant ACE-NME units 64, as shown in Equation 1, where n is the number of amino acids or data units.

$${E}_{t o t a {l}_{u n i t s}} = {\sum }_{i = 1}^{n} {E}_{data unit}^{i} - {\sum }_{i = 1}^{n - 1} {E}_{A C E - N M E}^{i} \qquad ( 1 )$$

The force for atoms in the same data unit 36 and ACE-NME 64 is calculated following Equation 2.

$${F}_{a t o {m}_{u n i t s}}^{i} = {\sum }_{j = 1}^{m} {F}_{data unit}^{i j} - {\sum }_{j = 1}^{n} {F}_{ACE - NME}^{ij} \qquad ( 2 )$$

In Eq. 2, i represents an atom for force calculation, m represents all the data units to which the atom i belongs, n represents all the ACE-NME units 64 to which the atom i belongs, and j represents any other atom that coexists with atom i in the same data unit 36 or ACE-NME unit 64.

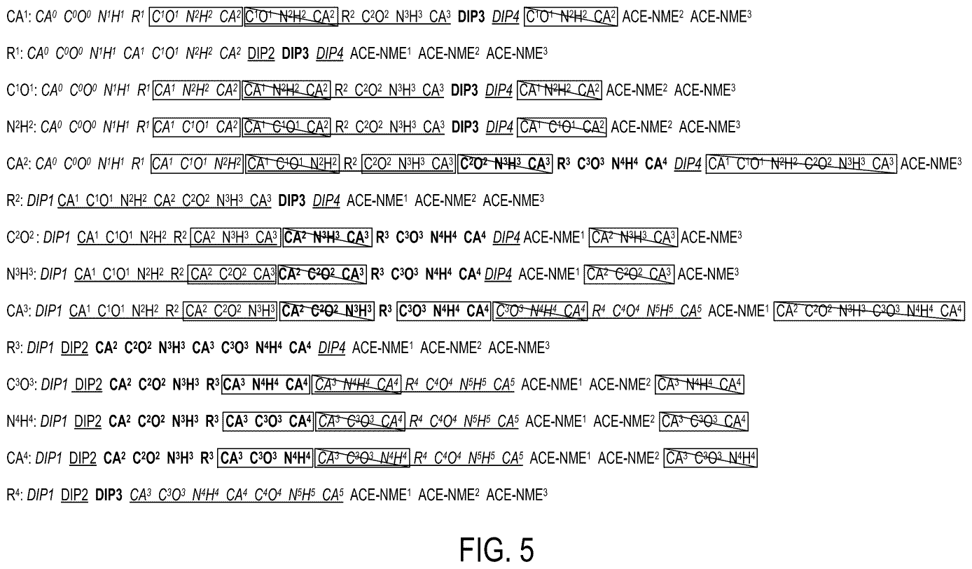

FIGS. 4 and 5 illustrate example atoms that coexist in the tetrapeptide introduced in FIG. 3 and discussed above. The tetrapeptide is illustrated again in FIG. 4 for reference. Atoms included in the data unit 36A, i.e., dipeptide 1 (DIP1), are indicated in italic; atoms included in the data unit 36B, i.e. dipeptide 2 (DIP2), are indicated in underline; atoms included in the data unit 36C, i.e., dipeptide 3 (DIP3), are indicated in bold; and atoms included in the data unit 36D, i.e., dipeptide 4 (DIP4), are indicated in italic and underline.

Looking at the first line of FIG. 4, the neighboring atoms that coexist with the alpha carbon CA¹ are shown. CA¹ coexists with atoms in the first data unit 36A and the second data unit 36B, as well as the first ACE-NME. The force for each atom pair of CA¹ and a coexisting atom are then calculated and summed. Atoms for data units and ACE-NMEs that do not coexist with the atom are not represented in atomic form. For example, CA¹ does not coexist atoms in data unit 36C (represented as DIP3), data unit 36D (DIP4), ACE-NME², or ACE-NME³.

Atoms included in duplicate regions are indicated by boxes, and atoms from all but one of the duplicate regions are removed from the calculation, as indicated by the crossed-out boxes. For example, the region including C¹O¹, N²H², and CA² is included in the first data unit 36A, the second data unit 36B, and the first ACE-NME unit. As such, the atoms in the second data unit 36B and the first ACE-NME unit are excluded from the calculation of force for atom pairs with CA¹. FIG. 4 shows a subset of atoms included the tetrapeptide, and FIG. 5 shows coexisting atoms for each of the atoms in data units 36A, 36B, 36C, 36D, and ACE-NME¹, ACE-NME², and ACE-NME³ of the tetrapeptide.

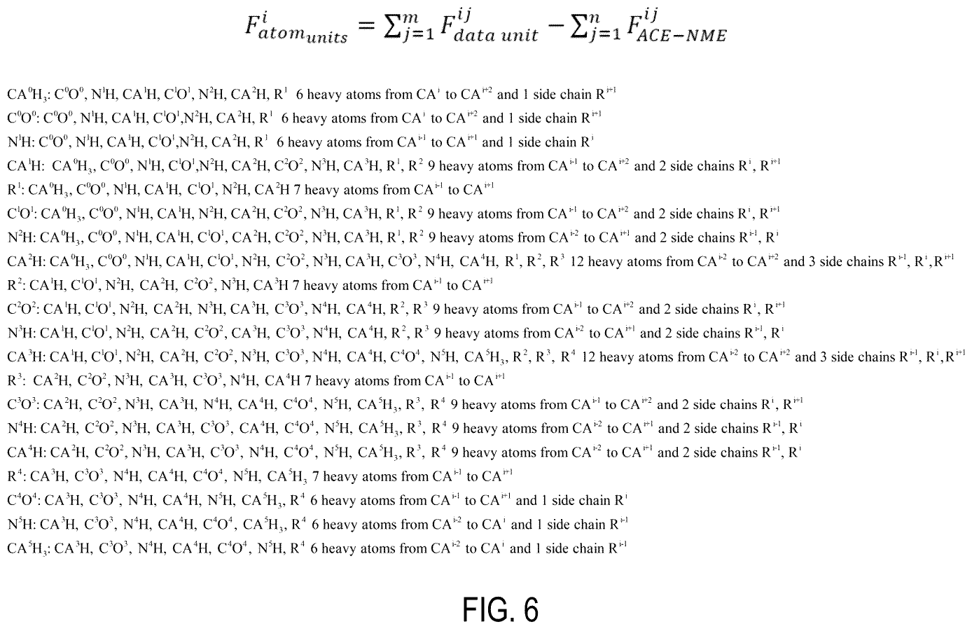

Coexisting atoms pairs for each atom in the tetrapeptide that will be included in the force calculation of the polypeptide are shown in FIG. 6. Following each line of coexisting atoms is a summary of the interactions. For example, in the first line of FIG. 6, CA⁰H₃ coexists with C⁰O⁰, NIH, CA¹H, C¹O¹, N²H, CA²H, R¹, which can be summarized as the six heavy atoms from CAⁱ to CAⁱ⁺² and one side chain Rⁱ⁺¹.

Alternatively, the data unit properties calculation module 42 may run an ML model 46 to calculate the force of each atom in each generated data unit 36, and to calculate the energy of each data unit. The ML model 46 may be implemented as a Vector-Scalar interactive Graph Neural Network (ViSNet), for example. With this approach, the coordinates and atom types for each data unit 36 or ACE-NME unit 64 are the input for the ViSNet model, and the model produces force for each atom and energy for the data unit 36.

### Polypeptide Properties Calculation

Using the quantum simulation program 44 or the ML model 46 described above enables calculation of all the energy and forces in the same data unit 36 and ACE-NME units 64. However, the extra interactions among different units have not been calculated. The following paragraphs provide additional description of implementations for calculating the molecular properties of extra interactions among the data units 36 and ACE-NME units 64 to determine the force and energy for the polypeptide sequence. As discussed above, there are two different ways to calculate the molecular properties of the polypeptide sequence 28, including a classic MM program 50 and a QM-MM simulation program 52.

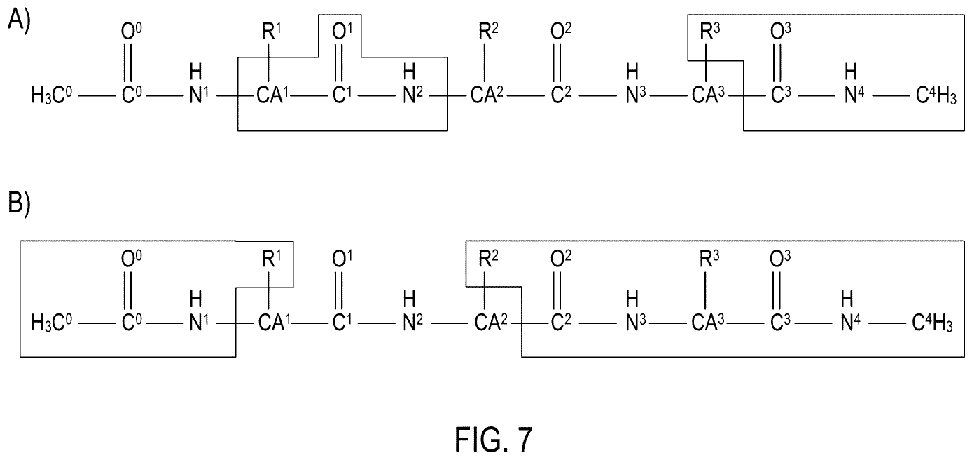

FIG. 7 shows atoms in the tetrapeptide (see FIGS. 3 and 4) for which extra interactions need to be calculated. The top panel A) of FIG. 7 illustrates the interactions between atoms of the first data unit 36A and atoms of the third data unit 36C that have not been calculated. Specifically, the boxed regions in the top panel A) indicate interactions between the CA¹, C¹O¹, N²H atoms of the first data unit 36A and the R³, C³O³, N⁴H, C⁴H₃ atoms of the third data unit 36C that need to be calculated.

The bottom panel B) of FIG. 7 illustrates the interactions between atoms of the first data unit 36A and atoms of the second and third data units 36B, 36C that have not been calculated. Specifically, the boxed regions in the bottom panel B) indicate interactions between the C⁰H₃, C⁰O⁰, N¹H, and R¹ atoms of the first data unit 36A and the R², C²O², N³H, CA³, R³, C³O³, N⁴H, C⁴H₃ atoms of the second and third data units 36B, 36C that need to be calculated. As discussed above and described in detail below, there are two approaches to estimate these interactions.

In the first approach, the extra interactions are calculated by MM. The MM approach includes of two kinds of interactions: Coulomb and van der Waals. Then, corresponding parameters from a molecular dynamics (MD) force field (FF) simulation program and the distance between atoms are used to calculate the energy and force, as shown in Equations 3 and 4, below.

$${E}_{t o t a l} = {E}_{{total}_{units}} + {\sum }_{\begin{matrix}i = 1 \\ i ∈ A\end{matrix}}^{n - 1} {\sum }_{\begin{matrix}j = i + 1 \\ j ∉ A\end{matrix}}^{n} {E}_{Coulomb}^{i j} + {\sum }_{\begin{matrix}i = 1 \\ i ∈ A\end{matrix}}^{n - 1} {\sum }_{\begin{matrix}j = i + 1 \\ j ∉ A\end{matrix}}^{n} {E}_{V D W}^{i j} \qquad ( 3 )$$

$${F}_{a t o {m}_{t o t a l}}^{i} = {F}_{a t o {m}_{u n i t s}}^{i} + {\sum }_{\begin{matrix}i = 1 \\ i ∈ A\end{matrix}}^{n - 1} {\sum }_{\begin{matrix}j = i + 1 \\ j ∉ A\end{matrix}}^{n} {F}_{C o u l o m b}^{i j} + {\sum }_{\begin{matrix}i = 1 \\ i ∈ A\end{matrix}}^{n - 1} {\sum }_{\begin{matrix}j = i + 1 \\ j ∉ A\end{matrix}}^{n} {F}_{V D W}^{i j} \qquad ( 4 )$$

The MD FF simulation program may be, for example, Assisted Model Building with Energy Refinement (AMBER), using the FF19SB force field that uses amino acid-specific backbone parameters and improves modeling of amino acid-dependent properties such as helical propensities.

The energy E and force F with subscript “units” represent the value obtained from the data unit 36 and ACE-NME unit 64 combination (Eq. 1, 2), and A indicates the atom set in each data unit 36. As shown in Eq. 4, the sum in the second and third terms traverse all the atoms j with the indices after the current atom i and do not coexist with atom i in any data units 36.

With the second approach, the extra interactions are calculated by a combination of QM and MM. In the QM-MM approach, the interactions of nearby side chains (i.e., side chains within a distance threshold λ of one another) are calculated via counterpoise correction by quantum simulation at the DFT level, and the interactions between the remaining atom pairs are calculated with the MM approach.

FIG. 8 shows atoms in the tetrapeptide (see FIGS. 3 and 4) for which extra interactions need to be calculated. The top panel A) of FIG. 8 illustrates the interactions between atoms of the first side chain R¹, the second side chain R², and the third side chain R³. A minimal distance between each side chain is determined. Interactions between side chains separated by a distance less than or equal to a threshold distance λ are calculated by counterpoise QM using DFT. As shown in the top panel A), the distance between R¹-R² is within the threshold distance λ, while the distances between R¹_R³ and R²-R³ are greater than the threshold distance λ. Thus, the interactions between the atoms in side chains R¹ and R² will be calculated via counterpoise correction by quantum simulation and DFT, as described below, and the interactions between R¹ and R³, and R² and R³ will be calculated using the MM approach described above.

The middle panel B) of FIG. 8 illustrates the extra interactions that were calculated using the MM approach. The interactions between atoms in the side chains are not included in this calculation, as those interactions were determined based on the threshold distance λ. The bottom panel C) of FIG. 8 illustrates the extra interactions between atoms that are calculated by MM. When using the MM approach as described in FIG. 7, these interactions include atoms in the R¹ side chain. However, when using the QM-MM approach, the interactions between atoms in the R¹ side chain are calculated using QM or MM, depending on the threshold distance λ, and are thus not included in the extra interactions calculated using MM as a default.

To build the counterpoise QM system, the coordinates of the two side chains included in the calculation are extracted, and a hydrogen is added to the beta carbon of the side chain in the direction of the alpha carbon, according to C—H bond length. If the side chain is glycine, the hydrogen is added according to the H—H bond length. If the side chain is proline, two hydrogens are added: one to the beta carbon in the direction of the alpha carbon, and one to the delta carbon in the direction of the N-terminus. Then, the two side chains are used to build three systems. The first system has two side chains and their basis function, the second system has the first side chain and the basis function of both side chains, and the third system has second side chain and the basis function of both side chains. The algorithm is illustrated in Equations 5 and 6, shown below, where the first side chain is A, the second side chain is B, and λ defines the distance between the side chains in the polypeptide sequence.

$${E}_{t o t a l} = {E}_{t o t a {l}_{u n i t s}} + {\sum }_{\vert {R}_{A} - {R}_{B} \vert \leq λ} ( {E}_{b A B}^{A B} - {E}_{b A B}^{A} - {E}_{b A B}^{B} ) + {\sum }_{\begin{matrix}i = 1 \\ i ∈ A\end{matrix}}^{n - 1} {\sum }_{\begin{matrix}j = i + 1 \\ \begin{matrix}i ∉ A & j ∉ B\end{matrix}\end{matrix}}^{n} {E}_{Coulomb}^{i j} + {\sum }_{\begin{matrix}i = 1 \\ i ∈ A\end{matrix}}^{n - 1} {\sum }_{\begin{matrix}j = i + 1 \\ \begin{matrix}i ∉ A & j ∉ B\end{matrix}\end{matrix}}^{n} {E}_{V D W}^{i j} \qquad ( 5 )$$

$${F}_{{atom}_{total}}^{i} = {F}_{{atom}_{units}}^{i} + {\sum }_{\begin{matrix}\vert {R}_{A} - {R}_{B} \vert \leq λ \\ i ∈ A , j ∈ B\end{matrix}} ( {F}_{b A B}^{A B} - {F}_{b A B}^{A} - {F}_{b A B}^{B} ) + {\sum }_{\begin{matrix}i = 1 \\ i ∈ A\end{matrix}}^{n - 1} {\sum }_{\begin{matrix}j = i + 1 \\ \begin{matrix}i ∉ A & j ∉ B\end{matrix}\end{matrix}}^{n} {F}_{C o u l o m b}^{i j} + {\sum }_{\begin{matrix}i = 1 \\ i ∈ A\end{matrix}}^{n - 1} {\sum }_{\begin{matrix}j = i + 1 \\ \begin{matrix}i ∉ A & j ∉ B\end{matrix}\end{matrix}}^{n} {F}_{V D W}^{i j} \qquad ( 6 )$$

In the energy E calculation of Eq. 5,

$${E}_{b A B}^{A B}$$

indicates the interaction of a first system having A and B side chains in the A and B basis function, while

$${E}_{b A B}^{A}$$

indicates the interaction of a second system having the A side chain in the A and B basis function. The sum

$${E}_{b A B}^{A B} - {E}_{b A B}^{A} - {E}_{b A B}^{B}$$

reflects the counterpoise energy. Other subscripts in the force F calculation shown in Eq. 6 have similar meanings.

FIG. 9 shows a flowchart of a method 900 for fragment-based quantum mechanical calculation of protein properties, according to one example implementation of the present disclosure. The method 900 may be implemented by the hardware and software of computing system 10 described above, or by other suitable hardware and software.

It will be appreciated that steps 902 through 910 of the method 900 are performed for each subsequence of three adjacent amino acids in a polypeptide sequence. At step 902, method 900 includes identifying a first amino acid. As described above, the first amino acid has a first main chain comprising a first amino group, a first alpha carbon, and a first carboxyl group, and a first side chain attached to the first alpha carbon.

Continuing from step 902 to step 904, the method 900 includes identifying a second amino acid. As described above, the second amino acid has a second main chain comprising a second amino group, a second alpha carbon, and a second carboxyl group, and a second side chain attached to the second alpha carbon.

Proceeding from step 904 to step 906, the method 900 includes identifying a third amino acid. As described above, the third amino acid has a third main chain comprising a third amino group, a third alpha carbon, and a third carboxyl group, and a third side chain attached to the third alpha carbon.

Advancing from step 906 to step 908, the method 900 includes generating a data unit. As described above, the data unit comprises data representing the first alpha carbon, the first carboxyl group, the second amino group, the second alpha carbon, the second carboxyl group, the second side chain, the third amino group, and the third alpha carbon. The first alpha carbon and the first carboxyl group comprise an N-terminal acetyl group (ACE) of the data unit, and the third amino group and the third alpha carbon comprise a C-terminal N-methylamino group (NME) of the data unit. The data unit further includes data representing a first peptide bond formed between the N-terminal ACE and the second amino group, and a second peptide bond formed between the second carboxyl group and the C-terminal NME. Together, the generated data units for the polypeptide sequence represent the atomic structure of the of amino acids in the polypeptide sequence.

The method may further include adding data representing one or more additional hydrogens to the first alpha carbon in each data unit according to a first bond length and a first direction of a previous bond between the first alpha carbon and the first side chain, and adding data representing one or more additional hydrogens to the third alpha carbon in each data unit according to a third bond length and a third direction of a previous bond between the third alpha carbon and the third side chain.

Continuing from step 908 to step 910, the method 900 includes storing the generated data unit in a database. The plurality of data units for the polypeptide sequence may be stored in a container in the database, and the database may include multiple containers that each store data units derived from a respective polypeptide sequence.

Proceeding from step 910 to step 912, the method 900 includes calculating a force of each atom in the data unit. Advancing from step 912 to step 914, the method 900 includes calculating an energy of the data unit. In a quantum mechanical mode, density functional theory is applied to calculate the force of each atom in the generated data unit, and to calculate the energy of the data unit. In a machine learning mode, coordinates and atom types for each data unit are input into a machine learning model to calculate the force of each atom in the generated data unit, and to calculate the energy of the data unit.

Continuing from step 914 to step 916, the method 900 includes calculating a force of the polypeptide sequence based on the calculated forces of each atom in each data unit of the plurality of data units. The energy of the polypeptide sequence is calculated by summing the calculated energy of each data unit of the plurality of data units and subtracting energies of duplicated regions shared by adjacent data units of the polypeptide sequence.

Proceeding from step 916 to step 918, the method 900 includes calculating an energy of the polypeptide sequence based on the calculated energies for each data unit of the plurality of data units. The force of the polypeptide sequence is calculated by summing the calculated force of each data unit of the plurality of data units and subtracting forces of duplicated regions shared by adjacent data units of the polypeptide sequence.

As described in detail above, interactions between main chain atoms of a data unit and side chain atoms of non-adjacent data units are calculated via molecular mechanics, interactions between side chain atoms of data units separated by a distance that is less than or equal to a distance threshold are calculated via counterpoise quantum mechanics applying DFT, and interactions between side chain atoms of data units separated by a distance that is greater than the distance threshold are calculated via molecular mechanics.

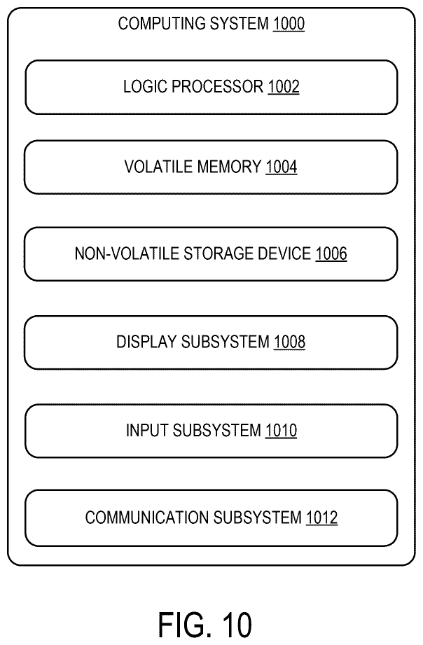

FIG. 10 schematically shows a non-limiting embodiment of a computing system 1000 that can enact one or more of the methods and processes described above. Computing system 1000 is shown in simplified form. Computing system 1000 may embody the computer system 10 described above and illustrated in FIG. 1. Computing system 1000 may take the form of one or more personal computers, server computers, tablet computers, home-entertainment computers, network computing devices, gaming devices, mobile computing devices, mobile communication devices (e.g., smart phone), and/or other computing devices, and wearable computing devices such as smart wristwatches and head mounted augmented reality devices.

Computing system 1000 includes a logic processor 1002 volatile memory 1004, and a non-volatile storage device 1006. Computing system 1000 may optionally include a display subsystem 1008, input subsystem 1010, communication subsystem 1012, and/or other components not shown in FIG. 10.

Logic processor 1002 includes one or more physical devices configured to execute instructions. For example, the logic processor may be configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result.

The logic processor may include one or more physical processors (hardware) configured to execute software instructions. Additionally or alternatively, the logic processor may include one or more hardware logic circuits or firmware devices configured to execute hardware-implemented logic or firmware instructions. Processors of the logic processor 1002 may be single-core or multi-core, and the instructions executed thereon may be configured for sequential, parallel, and/or distributed processing. Individual components of the logic processor optionally may be distributed among two or more separate devices, which may be remotely located and/or configured for coordinated processing. Aspects of the logic processor may be virtualized and executed by remotely accessible, networked computing devices configured in a cloud-computing configuration. In such a case, these virtualized aspects are run on different physical logic processors of various different machines, it will be understood.

Non-volatile storage device 1006 includes one or more physical devices configured to hold instructions executable by the logic processors to implement the methods and processes described herein. When such methods and processes are implemented, the state of non-volatile storage device 1006 may be transformed—e.g., to hold different data.

Non-volatile storage device 1006 may include physical devices that are removable and/or built in. Non-volatile storage device 1006 may include optical memory (e.g., CD, DVD, HD-DVD, Blu-Ray Disc, etc.), semiconductor memory (e.g., ROM, EPROM, EEPROM, FLASH memory, etc.), and/or magnetic memory (e.g., hard-disk drive, floppy-disk drive, tape drive, MRAM, etc.), or other mass storage device technology. Non-volatile storage device 1006 may include nonvolatile, dynamic, static, read/write, read-only, sequential-access, location-addressable, file-addressable, and/or content-addressable devices. It will be appreciated that non-volatile storage device 1006 is configured to hold instructions even when power is cut to the non-volatile storage device 1006.

Volatile memory 1004 may include physical devices that include random access memory. Volatile memory 1004 is typically utilized by logic processor 1002 to temporarily store information during processing of software instructions. It will be appreciated that volatile memory 1004 typically does not continue to store instructions when power is cut to the volatile memory 1004.

Aspects of logic processor 1002, volatile memory 1004, and non-volatile storage device 1006 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms “module,” “program,” and “engine” may be used to describe an aspect of computing system 1000 typically implemented in software by a processor to perform a particular function using portions of volatile memory, which function involves transformative processing that specially configures the processor to perform the function. Thus, a module, program, or engine may be instantiated via logic processor 1002 executing instructions held by non-volatile storage device 1006, using portions of volatile memory 1004. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

When included, display subsystem 1008 may be used to present a visual representation of data held by non-volatile storage device 1006. The visual representation may take the form of a graphical user interface (GUI). As the herein described methods and processes change the data held by the non-volatile storage device, and thus transform the state of the non-volatile storage device, the state of display subsystem 1008 may likewise be transformed to visually represent changes in the underlying data. Display subsystem 1008 may include one or more display devices utilizing virtually any type of technology. Such display devices may be combined with logic processor 1002, volatile memory 1004, and/or non-volatile storage device 1006 in a shared enclosure, or such display devices may be peripheral display devices.

When included, input subsystem 1010 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity; and/or any other suitable sensor. When included, communication subsystem 1012 may be configured to communicatively couple various computing devices described herein with each other, and with other devices. Communication subsystem 1012 may include wired and/or wireless communication devices compatible with one or more different communication protocols. As non-limiting examples, the communication subsystem may be configured for communication via a wireless telephone network, or a wired or wireless local- or wide-area network, such as a HDMI over Wi-Fi connection. In some embodiments, the communication subsystem may allow computing system 600 to send and/or receive messages to and/or from other devices via a network such as the Internet.

The following paragraphs provide additional description of aspects of the present disclosure. One aspect provides a computing system for fragment-based quantum mechanical calculation of protein properties. The computing system may comprise a processor that executes instructions using portions of associated memory to implement a protein fragmentation module that separates a computer-readable polypeptide sequence representing a plurality of amino acids into a plurality of data units. The protein fragmentation module may be configured to, for each subsequence of three adjacent amino acids in the polypeptide sequence: identify a first amino acid having a first main chain comprising a first amino group, a first alpha carbon, and a first carboxyl group, and a first side chain attached to the first alpha carbon; identify a second amino acid having a second main chain comprising a second amino group, a second alpha carbon, and a second carboxyl group, and a second side chain attached to the second alpha carbon; identify a third amino acid having a third main chain comprising a third amino group, a third alpha carbon, and a third carboxyl group, and a third side chain attached to the third alpha carbon; generate a data unit comprising data representing the first alpha carbon, the first carboxyl group, the second amino group, the second alpha carbon, the second carboxyl group, the second side chain, the third amino group, and the third alpha carbon; and store the generated data unit in a database.

In this aspect, additionally or alternatively, the first alpha carbon and the first carboxyl group may comprise an N-terminal acetyl group (ACE) of the data unit, the third amino group and the third alpha carbon may comprise a C-terminal N-methylamino group (NME) of the data unit, and the data unit may further include a first peptide bond formed between the N-terminal ACE and the second amino group, and a second peptide bond formed between the second carboxyl group and the C-terminal NME.

In this aspect, additionally or alternatively, data representing one or more additional hydrogens may be added to the first alpha carbon in each data unit according to a first bond length and a first direction of a previous bond between the first alpha carbon and the first side chain, and data representing one or more additional hydrogens may be added to the third alpha carbon in each data unit according to a third bond length and a third direction of a previous bond between the third alpha carbon and the third side chain.

In this aspect, additionally or alternatively, a limited-memory Broyden-Fletcher-Goldfarb-Shanno quasi-Newton (LBFGS) algorithm may be applied to optimize the position of the one or more additional hydrogens.

In this aspect, additionally or alternatively, the processor may be further configured to execute instructions to implement a data unit properties calculation module that calculates a force of each atom in the data unit, and calculates an energy of the data unit.

In this aspect, additionally or alternatively, in a quantum mechanical (QM) mode, the data unit properties calculation module may apply density functional theory (DFT) to calculate the force of each atom in the generated data unit, and to calculate the energy of the data unit.

In this aspect, additionally or alternatively, in a machine learning mode, the data unit properties calculation module may input coordinates and atom types for each data unit into a machine learning model to calculate the force of each atom in the generated data unit, and to calculate the energy of the data unit.

In this aspect, additionally or alternatively, the processor may be further configured to execute instructions to implement a polypeptide properties calculation module. The polypeptide properties calculation module may calculate a force of the polypeptide sequence based on the calculated forces of each atom in each data unit of the plurality of data units, and may calculate an energy of the polypeptide sequence based on the calculated energies for each data unit of the plurality of data units. The energy of the polypeptide sequence may be calculated by summing the calculated energy of each data unit of the plurality of data units and subtracting energies of duplicated regions shared by adjacent data units of the polypeptide sequence. The force of the polypeptide sequence may be calculated by summing the calculated force of each data unit of the plurality of data units and subtracting forces of duplicated regions shared by adjacent data units of the polypeptide sequence.

In this aspect, additionally or alternatively, interactions between main chain atoms of a data unit and side chain atoms of non-adjacent data units may be calculated via molecular mechanics.

In this aspect, additionally or alternatively, interactions between side chain atoms of data units separated by a distance that is less than or equal to a distance threshold may be calculated via counterpoise quantum mechanics applying DFT, and interactions between side chain atoms of data units separated by a distance that is greater than the distance threshold may be calculated via molecular mechanics.

Another aspect provides a method for fragment-based quantum mechanical calculation of protein properties. The method may comprise, for each subsequence of three adjacent amino acids in a polypeptide sequence, identifying a first amino acid, the first amino acid having a first main chain comprising a first amino group, a first alpha carbon, and a first carboxyl group, and a first side chain attached to the first alpha carbon; identifying a second amino acid, the second amino acid having a second main chain comprising a second amino group, a second alpha carbon, and a second carboxyl group, and a second side chain attached to the second alpha carbon; identifying a third amino acid, the third amino acid having a third main chain comprising a third amino group, a third alpha carbon, and a third carboxyl group, and a third side chain attached to the third alpha carbon; generating a data unit comprising data representing the first alpha carbon, the first carboxyl group, the second amino group, the second alpha carbon, the second carboxyl group, the second side chain, the third amino group, and the third alpha carbon; and storing the generated data unit in a database.

In this aspect, additionally or alternatively, the first alpha carbon and the first carboxyl group may comprise an N-terminal acetyl group (ACE) of the data unit, the third amino group and the third alpha carbon may comprise a C-terminal N-methylamino group (NME) of the data unit, and the data unit may further include data representing a first peptide bond formed between the N-terminal ACE and the second amino group, and a second peptide bond formed between the second carboxyl group and the C-terminal NME.

In this aspect, additionally or alternatively, the method may further comprise adding data representing one or more additional hydrogens to the first alpha carbon in each data unit according to a first bond length and a first direction of a previous bond between the first alpha carbon and the first side chain, and adding data representing one or more additional hydrogens to the third alpha carbon in each data unit according to a third bond length and a third direction of a previous bond between the third alpha carbon and the third side chain.

In this aspect, additionally or alternatively, the method may further comprise calculating a force of each atom in the data unit, and calculating an energy of the data unit.

In this aspect, additionally or alternatively, the method may further comprise, in a quantum mechanical mode, applying density functional theory to calculate the force of each atom in the generated data unit, and to calculate the energy of the data unit.

In this aspect, additionally or alternatively, the method may further comprise, in a machine learning mode, inputting coordinates and atom types for each data unit into a machine learning model to calculate the force of each atom in the generated data unit, and to calculate the energy of the data unit.

In this aspect, additionally or alternatively, the method may further comprise calculating a force of the polypeptide sequence based on the calculated forces of each atom in each data unit of the plurality of data units, and calculating an energy of the polypeptide sequence based on the calculated energies for each data unit of the plurality of data units. The energy of the polypeptide sequence may be calculated by summing the calculated energy of each data unit of the plurality of data units and subtracting energies of duplicated regions shared by adjacent data units of the polypeptide sequence. The force of the polypeptide sequence may be calculated by summing the calculated force of each data unit of the plurality of data units and subtracting forces of duplicated regions shared by adjacent data units of the polypeptide sequence.

In this aspect, additionally or alternatively, the method may further comprise calculating interactions between main chain atoms of a data unit and side chain atoms of non-adjacent data units via molecular mechanics.

In this aspect, additionally or alternatively, the method may further comprise calculating interactions between side chain atoms of data units separated by a distance that is less than or equal to a distance threshold via counterpoise quantum mechanics applying DFT, and calculating interactions between side chain atoms of data units separated by a distance that is greater than the distance threshold via molecular mechanics.

Another aspect provides a computing system for fragment-based quantum mechanical calculation of protein properties. The computing system may comprise a processor that executes instructions using portions of associated memory to implement a protein fragmentation module that separates a computer-readable polypeptide sequence representing a plurality of amino acids into a plurality of data units. The protein fragmentation module may be configured to, for each subsequence of three adjacent amino acids in the polypeptide sequence, generate a data unit comprising data representing a first alpha carbon and a first carboxyl group from a first amino acid, a second amino group, a second alpha carbon, a second carboxyl group, and a second side chain from a second amino acid, and a third amino group and a third alpha carbon of a third amino acid. Using a quantum simulation program, a data unit properties calculation module may apply density functional theory (DFT) to calculate the force of each atom in the generated data unit, and to calculate the energy of the data unit. A polypeptide properties calculation module may calculate a force of the polypeptide sequence based on the calculated forces of each atom in each data unit of the plurality of data units, and may calculate an energy of the polypeptide sequence based on the calculated energies for each data unit of the plurality of data units.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1-15. (canceled)

16. A computing system for fragment-based quantum mechanical calculation of protein properties, comprising:
a processor that executes instructions using portions of associated memory to implement a protein fragmentation module that separates a computer-readable polypeptide sequence representing a plurality of amino acids into a plurality of data units, wherein
the protein fragmentation module is configured to, for each subsequence of three adjacent amino acids in the polypeptide sequence:
identify a first amino acid having a first main chain comprising a first amino group, a first alpha carbon, and a first carboxyl group, and a first side chain attached to the first alpha carbon,
identify a second amino acid having a second main chain comprising a second amino group, a second alpha carbon, and a second carboxyl group, and a second side chain attached to the second alpha carbon,
identify a third amino acid having a third main chain comprising a third amino group, a third alpha carbon, and a third carboxyl group, and a third side chain attached to the third alpha carbon,
generate a data unit comprising data representing the first alpha carbon, the first carboxyl group, the second amino group, the second alpha carbon, the second carboxyl group, the second side chain, the third amino group, and the third alpha carbon, and
store the generated data unit in a database.

17. The computing system of claim 16, wherein
the first alpha carbon and the first carboxyl group comprise an N-terminal acetyl group (ACE) of the data unit,
the third amino group and the third alpha carbon comprise a C-terminal N-methylamino group (NME) of the data unit, and
the data unit further includes a first peptide bond formed between the N-terminal ACE and the second amino group, and a second peptide bond formed between the second carboxyl group and the C-terminal NME.

18. The computing system of claim 16, wherein
data representing one or more additional hydrogens is added to the first alpha carbon in each data unit according to a first bond length and a first direction of a previous bond between the first alpha carbon and the first side chain, and
data representing one or more additional hydrogens is added to the third alpha carbon in each data unit according to a third bond length and a third direction of a previous bond between the third alpha carbon and the third side chain.

19. The computing system of claim 18, wherein
a limited-memory Broyden-Fletcher-Goldfarb-Shanno quasi-Newton (LBFGS) algorithm is applied to optimize the position of the one or more additional hydrogens.

20. The computing system of claim 16, wherein the processor is further configured to execute instructions to implement:
a data unit properties calculation module that calculates a force of each atom in the data unit, and calculates an energy of the data unit.

21. The computing system of claim 20, wherein
in a quantum mechanical (QM) mode, the data unit properties calculation module applies density functional theory (DFT) to calculate the force of each atom in the generated data unit, and to calculate the energy of the data unit.

22. The computing system of claim 20, wherein
in a machine learning mode, the data unit properties calculation module inputs coordinates and atom types for each data unit into a machine learning model to calculate the force of each atom in the generated data unit, and to calculate the energy of the data unit.

23. The computing system of claim 20, wherein the processor is further configured to execute instructions to implement:
a polypeptide properties calculation module that calculates a force of the polypeptide sequence based on the calculated forces of each atom in each data unit of the plurality of data units, and calculates an energy of the polypeptide sequence based on the calculated energies for each data unit of the plurality of data units, wherein
the energy of the polypeptide sequence is calculated by summing the calculated energy of each data unit of the plurality of data units and subtracting energies of duplicated regions shared by adjacent data units of the polypeptide sequence, and
the force of the polypeptide sequence is calculated by summing the calculated force of each data unit of the plurality of data units and subtracting forces of duplicated regions shared by adjacent data units of the polypeptide sequence.

24. The computing system of claim 23, wherein
interactions between main chain atoms of a data unit and side chain atoms of non-adjacent data units are calculated via molecular mechanics.

25. The computing system of claim 23, wherein
interactions between side chain atoms of data units separated by a distance that is less than or equal to a distance threshold are calculated via counterpoise quantum mechanics applying DFT, and
interactions between side chain atoms of data units separated by a distance that is greater than the distance threshold are calculated via molecular mechanics.

26. A method for fragment-based quantum mechanical calculation of protein properties, the method comprising:
for each subsequence of three adjacent amino acids in a polypeptide sequence:
identifying a first amino acid, the first amino acid having a first main chain comprising a first amino group, a first alpha carbon, and a first carboxyl group, and a first side chain attached to the first alpha carbon;
identifying a second amino acid, the second amino acid having a second main chain comprising a second amino group, a second alpha carbon, and a second carboxyl group, and a second side chain attached to the second alpha carbon;
identifying a third amino acid, the third amino acid having a third main chain comprising a third amino group, a third alpha carbon, and a third carboxyl group, and a third side chain attached to the third alpha carbon;
generating a data unit comprising data representing the first alpha carbon, the first carboxyl group, the second amino group, the second alpha carbon, the second carboxyl group, the second side chain, the third amino group, and the third alpha carbon; and
storing the generated data unit in a database.

27. The method of claim 26, wherein
the first alpha carbon and the first carboxyl group comprise an N-terminal acetyl group (ACE) of the data unit,
the third amino group and the third alpha carbon comprise a C-terminal N-methylamino group (NME) of the data unit, and
the data unit further includes data representing a first peptide bond formed between the N-terminal ACE and the second amino group, and a second peptide bond formed between the second carboxyl group and the C-terminal NME.

28. The method of claim 26, the method further comprising:
adding data representing one or more additional hydrogens to the first alpha carbon in each data unit according to a first bond length and a first direction of a previous bond between the first alpha carbon and the first side chain; and
adding data representing one or more additional hydrogens to the third alpha carbon in each data unit according to a third bond length and a third direction of a previous bond between the third alpha carbon and the third side chain.

29. The method of claim 26, the method further comprising:
calculating a force of each atom in the data unit; and
calculating an energy of the data unit.

30. The method of claim 29, the method further comprising:
in a quantum mechanical mode, applying density functional theory to calculate the force of each atom in the generated data unit, and to calculate the energy of the data unit.

31. The method of claim 29, the method further comprising:
in a machine learning mode, inputting coordinates and atom types for each data unit into a machine learning model to calculate the force of each atom in the generated data unit, and to calculate the energy of the data unit.

32. The method of claim 29, the method further comprising:
calculating a force of the polypeptide sequence based on the calculated forces of each atom in each data unit of the plurality of data units; and
calculating an energy of the polypeptide sequence based on the calculated energies for each data unit of the plurality of data units, wherein
the energy of the polypeptide sequence is calculated by summing the calculated energy of each data unit of the plurality of data units and subtracting energies of duplicated regions shared by adjacent data units of the polypeptide sequence, and
the force of the polypeptide sequence is calculated by summing the calculated force of each data unit of the plurality of data units and subtracting forces of duplicated regions shared by adjacent data units of the polypeptide sequence.

33. The method of claim 32, the method further comprising:
calculating interactions between main chain atoms of a data unit and side chain atoms of non-adjacent data units via molecular mechanics.

34. The method of claim 32, the method further comprising:
calculating interactions between side chain atoms of data units separated by a distance that is less than or equal to a distance threshold via counterpoise quantum mechanics applying DFT; and
calculating interactions between side chain atoms of data units separated by a distance that is greater than the distance threshold via molecular mechanics.

35. A computing system for fragment-based quantum mechanical calculation of protein properties, comprising:
a processor that executes instructions using portions of associated memory to implement a protein fragmentation module that separates a computer-readable polypeptide sequence representing a plurality of amino acids into a plurality of data units, wherein
the protein fragmentation module is configured to, for each subsequence of three adjacent amino acids in the polypeptide sequence, generate a data unit comprising data representing:
a first alpha carbon and a first carboxyl group from a first amino acid;
a second amino group, a second alpha carbon, a second carboxyl group, and a second side chain from a second amino acid; and
a third amino group and a third alpha carbon of a third amino acid,
using a quantum simulation program, a data unit properties calculation module applies density functional theory (DFT) to calculate the force of each atom in the generated data unit, and to calculate the energy of the data unit, and
a polypeptide properties calculation module calculates a force of the polypeptide sequence based on the calculated forces of each atom in each data unit of the plurality of data units, and calculates an energy of the polypeptide sequence based on the calculated energies for each data unit of the plurality of data units.
